(19) United States
(12) Patent Application Publication
(10) Pub. No.: US 2002/0042648 A1
(43) Pub. Date: Apr. 11, 2002

(54) Stent

(75) Inventors: Max Schaldach, Erlangen (DE); Daniel Lootz, Warnemuende (DE); Karsten Koop, Rostock (DE); Curt Kranz, Berlin (DE); Max Schaldach JR., Berlin (DE)

Correspondence Address: HAHN LOESER & PARKS, LLP, TWIN OAKS ESTATE, 1225 W. MARKET STREET, AKRON, OH, 44313, US

(73) Assignee: Biotronik Mess- und Therapiegeraete GmbH & Co.

(21) Appl. No.: 09/974,777

(22) Filed: Oct. 9, 2001

(30) Foreign Application Priority Data
Oct. 10, 2000 (DE) 100 50 970.3

(51) Int. Cl. A61F 2/06

(52) U.S. Cl. 623/1.15

(57) ABSTRACT

A stent, in particular a coronary stent, for expansion from a first condition into an expanded second condition in which it holds a vessel in an expanded state, comprising a tubular body whose peripheral surface (1) is formed by a number of annular support portions (2) comprising bar elements (3) which are connected in the longitudinal direction of the stent by way of connecting bars (4), wherein the bar elements (3) of at least a first support portion (2, 2.1) extend in a meander configuration in the peripheral direction of the stent and the bar element portions (3.1, 3.2) adjoining a turning point (5) are arranged in a V-shape in the first condition of the stent, and wherein the bar element portions (3.1, 3.2) of the first support portion (2.1) extend curvedly in a first direction (7) in the longitudinal direction of the stent.

The present invention concerns a stent, in particular a coronary stent, for expansion from a first condition into an expanded second condition in which it holds a vessel in an expanded state, comprising a tubular body whose peripheral surface is formed by a number of annular support portions comprising bar elements which are connected in the longitudinal direction of the stent by way of connecting bars. In that arrangement the bar elements of at least a first support portion extend in a meander configuration in the peripheral direction of the stent and the bar element portions adjoining a turning point of the first support portion are arranged in a V-shape in the first condition of the stent.

### BACKGROUND OF THE ART

A stent is what is known as an intraluminal expansion element which is used to hold a vessel, for example a blood vessel, in the human or animal body, in an expanded state. For that purpose the stent is moved in a compressed first condition by means of a suitable catheter to the location in the vessel, which is to be held in the expanded state. When the implantation location is reached the stent is radially expanded into an expanded second condition. In the case of what are known as balloon-expansible stents the stent is expanded by means of a balloon catheter to such a degree that, by virtue of plastic deformation, even after removal of the balloon, it maintains its expanded second condition and thus supports the vessel. In the case of the stents which are referred to as self-expanding the stent is held in a compressed first condition against a return force, for example by means of a sheathing catheter. That constriction is released at the implantation location so that the stent of its own accord assumes its expanded second condition.

In both alternative configurations the stent has to be moved to the implantation location on the one hand through vessel configurations which are curved to greater or lesser degrees. The implantation location on the other hand itself involves a configuration which is curved to a greater or lesser extent. In order to provide that the stent can be moved to the implantation location as easily as possible and in order to ensure that it adapts as well as possible to the configuration of the vessel in the region of the implantation location, it is desirable for the stent to involve the highest possible degree of flexibility in relation to its longitudinal axis. Good flexibility is required in particular also when the stent in use is required to perform considerable movements with the vessel, as is the situation for example in the case of coronary stents or in many peripheral uses.

Flexibility is usually achieved in the known stents by virtue of a specific configuration of the connecting bars. Thus, German utility model DE 297 08 689 U1 discloses a stent of the general kind set forth, with bar elements extending in a meander configuration therearound. In that case, the bar element portions which extend in a V-shape from a reversal or turning point are of a rectilinear configuration so that the result is support portions which are relatively stiff in the longitudinal direction of the stent. A certain degree of flexibility of the stent in relation to its longitudinal axis is ensured by the S-shaped configuration of the connecting bars between certain annular support portions. The flexibility in that case arises out of the fact that the S-shaped connecting bars stretch under a tensile force and thus correspondingly increase in length.

The known stents however suffer from the disadvantage that, by virtue of the relatively great spacing between the regions with the above-mentioned S-shaped connecting bars and the limited deformability of the connecting bars, they can only follow relatively slightly curved vessel configurations. Furthermore they can only approach the curvature of the vessel in the manner of a polygonal configuration and therefore the snugness of their fit to the natural configuration of the vessel is only relatively poor.

### SUMMARY OF THE INVENTION

Therefore the object of the present invention is to provide a stent of the general kind set forth in the opening part of this specification, which does not suffer from the above-indicated disadvantages or suffers therefrom only to a lesser degree, and which in particular has improved flexibility in relation to its longitudinal axis.

Based on a stent as set forth in the classifying portion of claim 1, that object is attained by the features recited in the characterizing portion of claim 1.

The present invention is based on the technical teaching that a stent which is particularly flexible and which fits with a good snug fit to curved vessel configurations is obtained if not only the connecting bars but also the bar elements of the support portions, by virtue of a suitable configuration, themselves contribute to the flexibility of the stent. In accordance with the invention for that purpose the bar element portions of the first support portion extend curvedly in a first direction in the longitudinal direction of the stent.

In that case, the curvature of the bar element portions in the same direction provides that the bar element, in the longitudinal direction of the stent, is of a lower degree of stiffness than in the known stents with rectilinear bar element portions. Under the effect of a pressure force which is operative in the longitudinal direction of the stent the V-shape of the bar element portions can simply be compressed, in the longitudinal direction of the stent, by an increase in the degree of curvature of the bar element portions, and can thus also contribute to the flexibility of the stent. Likewise the bar element portions can stretch by a given amount under the effect of a tensile force operative in the longitudinal direction of the stent, and thereby also contribute to the flexibility of the stent.

The bar element portions can comprise a plurality of straight segments which extend inclinedly relative to each other to produce the curvature of the bar element portion in question. Typically the bar element portions are of a continuously curved configuration in order to achieve a stress distribution which is as uniform as possible and a low level of notch effect in the bar element portions.

In that case, the curvature of the bar elements may vary over their length in order to achieve adaptation to the stress distribution to be expected, in other words, to distribute the stresses as uniformly as possible. In other variants which are particularly simple to manufacture the bar element portions are curved uniformly over their length.

Commonly, the stent very substantially comprises first support portions in order to make the advantages thereof available over the entire length of the stent. It will be appreciated however that the stent may also comprise in a portion-wise manner bar elements which are of a different configuration and which do not involve the above-mentioned properties.

Many variants of the stent according to the invention are distinguished in that the bar element portions are curved in such a way, and additionally or alternatively the width of the bar elements varies over the length thereof in such a way, that the stresses which occur upon flexural deformation of the stent with respect to its longitudinal axis upon being moved to the implantation location remain below the plastic deformation limit of the stent material. In that case, the required curvature or distribution in respect of width can be ascertained on the basis of the forces which are to be expected during implantation and in use, and the stresses resulting therefrom in the bar elements. That design configuration ensures that the stent does not already experience a—possibly multiple plastic deformation upon being moved to the implantation location, which deformation under some circumstances can result in rupture, which is to be avoided at any case, upon expansion of the stent or in subsequent use thereof.

Advantageous developments of the stent according to the invention provide a number of adjacent first support portions whose bar element portions are curved in the same direction. Alternatively however it is also possible to provide a number of adjacent first support portions in which the direction of curvature of the bar element portions of the support portions changes in the longitudinal direction of the stent. In that case the direction of curvature of the bar element portions can again change from one support portion to another. Alternatively the support portions may have at least in paired fashion bar element portions involving the same direction of curvature. In other words, two or more support portions with the same first direction of curvature of the bar element portions are followed in the longitudinal direction of the stent by two or more support portions which involve a second direction of curvature, in opposite relationship to the first direction, of the bar element portions. It will be appreciated however that any other patterns of change in regard to the direction of curvature are also possible.

In particularly advantageous variants of the stent according to the invention the connecting bars are adapted and arranged to compensate for the reduction in length of the bar elements in the longitudinal direction of the stent upon expansion of the stent. For that purpose the connecting bars can be of such a configuration that, as a result of their deformation, upon expansion, there is an increase in the spacing between their engagement points on the two bar elements, which is sufficient to compensate for the reduction in length in the longitudinal direction, which results from stretching of the meandering bar elements in the peripheral direction. Thus for example curved connecting bars can engage the bar elements in such a way that, upon expansion of the stent, they are subjected to a stretching effect operative in the longitudinal direction of the stent.

In many cases, the engagement points and the length of the connecting bars are so selected that the reduction in length of the bar elements in the longitudinal direction of the stent, upon expansion of the stent, is substantially compensated. In this respect, the configuration is usually so selected that, with a substantially unaltered spacing between the two engagement points, the inclination of the connecting line between the two engagement points increases with respect to the peripheral direction upon expansion, in order to compensate for the reduction in length. If the engagement point is in the region of a reversal or turning point of the bar element in question, then the change in angle is correspondingly less, the closer that the respective engagement point is positioned in relation to the turning or reversal point. Therefore, to compensate for the reduction in length, the spacing between the engagement points must be selected to be appropriately great.

Thus in a variant which is particularly advantageous because it is simple to manufacture the connecting bars are of a substantially rectilinear configuration and extend between two mutually facing turning points of two adjoining bar elements, which in the first condition of the stent are displaced relative to each other in the peripheral direction of the stent by between one and two periods, usually by 1.5 times a period.

Alternatively however the connecting bars may also engage the central region of the respective bar element between the turning points, with the connecting line of the engagement points then extending substantially in the longitudinal direction of the stent.

Advantageous developments of the stent according to the invention are further distinguished in that the connecting bars are adapted to increase the flexibility of the stent. That can be effected in known manner. Thus, just by virtue of the length of the connecting bars, the above-described configuration with the long straight connecting bars between turning points which are displaced in the peripheral direction already contributes to the flexibility aspect. Alternatively a configuration for the connecting bars, which is correspondingly curved in an arcuate shape or a S-shape can also contribute to increasing flexibility.

In many variants of the stent according to the invention there are provided first connecting bars which involve a V-shaped configuration. On the one hand they increase the flexibility of the stent in the above-described manner while on the other hand they make it possible to achieve an improved supporting action, because it is over a larger surface area, while being simple to manufacture. That applies in particular in relation to many variants in which the first bars engage in the center region of the bar element portions and are of a configuration which is adapted to the curvature of the bar element portions. In other words, the configuration is selected to be such that the connecting bars substantially follow at a spacing the configuration of the bar element portions over a large part of their length.

The present invention further concerns an arrangement comprising a catheter for stent implantation purposes having a stent as set forth in hereinbefore. Depending on the respective nature of the stent this may involve a balloon catheter on which the stent is fitted, for example by crimping. It may likewise involve a sheathing catheter in which a stent in the form of a self-expanding stent is held in its first condition.

### BRIEF DESCRIPTION OF THE DRAWINGS

Further configurations of the present invention are set forth in the appendant claims and the description hereinafter of preferred variants of the stent according to the invention, with reference to the accompanying drawing in which:

FIG. 1 is a plan view of the development of the peripheral surface of a preferred embodiment of the stent according to the invention,

FIG. 2 is a plan view of the development of the peripheral surface of a further preferred embodiment of the stent according to the invention,

FIG. 3 is a plan view of the development of the peripheral surface of another preferred embodiment of the stent according to the invention, and

FIG. 4 is a plan view of the development of the peripheral surface of a further preferred embodiment of the stent according to the invention.

### DETAILED DESCRIPTION OF THE INVENTION

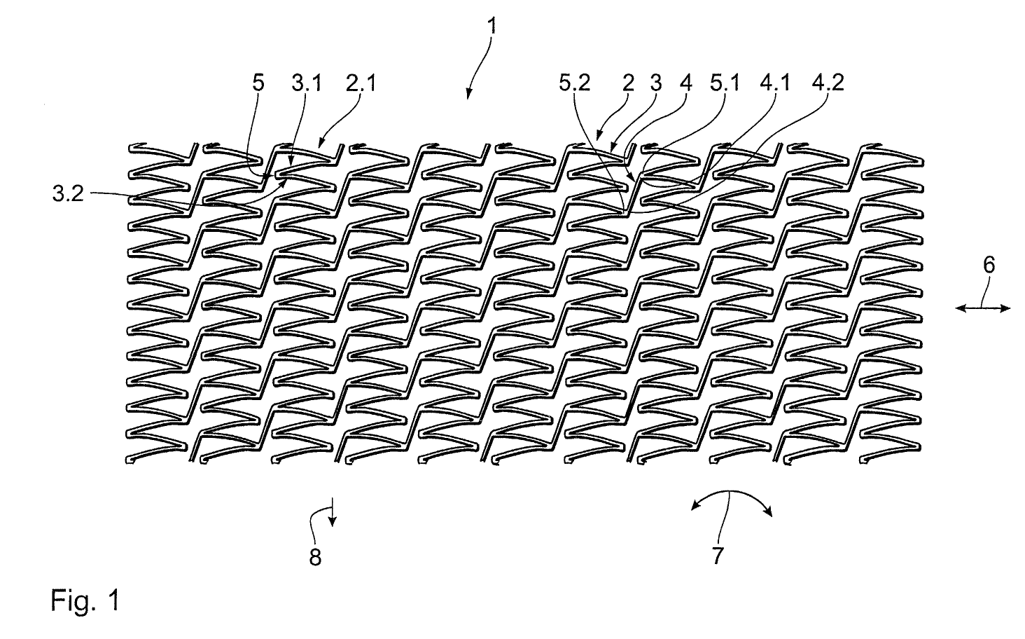

FIG. 1 is a plan view of the development of the peripheral surface 1 of an embodiment of a stent according to the invention, with a number of annular support portions 2. The development of the peripheral surface 1 is shown in the first condition of the stent, in which it can be introduced into the blood vessel. In the illustrated example the stent consists exclusively of first support portions 2.1 which are formed by bar elements 3 extending in a meander configuration in the peripheral direction of the stent. The bar elements 3 are connected together in the longitudinal direction of the stent by way of connecting bars 4.

The bar element portions 3.1 and 3.2 adjoining a turning or reversal point 5 are arranged in a V-shape in the illustrated first condition of the stent. They extend curvedly in a first direction indicated by the double-headed arrow 7, in the longitudinal direction of the stent as indicated by the double-headed arrow 6. By virtue of that curved, V-shaped arrangement of the bar element portions 3.1, 3.2 in the first condition of the stent, the bar elements 3 themselves contribute to flexibility of the stent, in relation to its longitudinal direction.

If for example a compression force is applied to the bar elements 3 in the longitudinal direction, the curvature of the bar element portions 3.1 and 3.2 increases, with the result that the bar element 3 is reduced in length in the longitudinal direction of the stent. If conversely a pulling force is applied to the bar elements 3 in the longitudinal direction, the curvature of the bar element portions 3.1 and 3.2 is reduced, with the result that the length of the bar element 3 increases in the longitudinal direction of the stent. If therefore the stent is to be bent or curved, for example to follow a curved vessel configuration, then the bar elements 3 are reduced in length in the longitudinal direction on the side of the stent which faces towards the center point of the curvature, by virtue of the compression force acting there, in the above-described manner, while, on its side remote from the associated center point of the curvature, the bar elements are increased in length in the longitudinal direction, by virtue of the pulling force acting there, in the above-described manner.

In the illustrated embodiment, the bar element portions 3.1 and 3.2 are of a continuous curvature which is uniform over the length thereof. That means that manufacture of the stent is particularly simple, by virtue of the simple geometry involved, with a desirable distribution of stresses over the bar element.

The curvature of the bar element portions 3.1 and 3.2 is so selected that the stresses which occur upon flexural deformation of the stent in relation to its longitudinal axis when it is being moved to the implantation location remain below the plastic deformation limit of the stent material. It will be appreciated that in other variants the width of the bar elements may also vary over the length thereof in order to achieve that effect.

In the illustrated embodiment the bar elements of all support portions 3 are respectively curved in the same direction 7. It will be appreciated however that in other variants a change in the direction of curvature of the bar element portions may also occur, between the support portions which are in mutually adjoining relationship in the longitudinal direction of the stent. It will further be appreciated that in a portion-wise manner the stent may also comprise bar elements of a different configuration, which do not involve the above-indicated properties.

The connecting bars 4 are adapted and arranged to compensate for the reduction in length of the bar elements 3 upon expansion of the stent into its second condition. For that purpose they are of a rectilinear nature and engage the region of mutually facing turning points 5.1 and 5.2 of two mutually adjoining bar elements 3, wherein the turning point 5.2 is displaced with respect to the turning point 5.1 by 1.5 times the period of the bar elements 3, in a first peripheral direction 8.

Upon expansion of the stent, by virtue of the change in angle occurring due to deformation, in the region of the respective turning point 5.1 and 5.2 respectively, the angle of inclination of the straight line connecting the engagement points 4.1 and 4.2 of the connecting bars 4 increases, in relation to the peripheral direction. That results in an increase in the spacing of the engagement points 4.1 and 4.2 of the connecting bars 4 in the longitudinal direction of the stent, whereby once again the reduction in length of the bar elements in the longitudinal direction of the stent upon expansion thereof is compensated.

In the illustrated embodiment the connecting bars 4 engage each second turning point 5 of the bar elements 3, in the peripheral direction. It will be appreciated that other variants may also involve a different number of connecting bars. In particular, a connecting bar may engage at each turning point. Equally however there may be larger spacings between the connecting bars in the peripheral direction.

In this respect, the length of the connecting bars 4 and the position of their engagement points 4.1 and 4.2 is so selected as to afford complete compensation for the reduction in length of the bar elements upon expansion of the stent into its second condition.

The length and the arrangement, inclinedly with respect to the longitudinal direction of the stent, of the connecting bars 4, further contributes to increasing the flexibility of the stent as just relatively low levels of pulling or compression forces operative in the longitudinal direction of the stent, as a result of the long lever arm, already result in considerable deflections of the connecting bars 4 in the longitudinal direction of the stent.

The stent shown in FIG. 1 is distinguished by a particularly high level of flexibility and by particular ease of manufacture, by virtue of its simple geometry. It can be particularly easily and well crimped on to a balloon catheter.

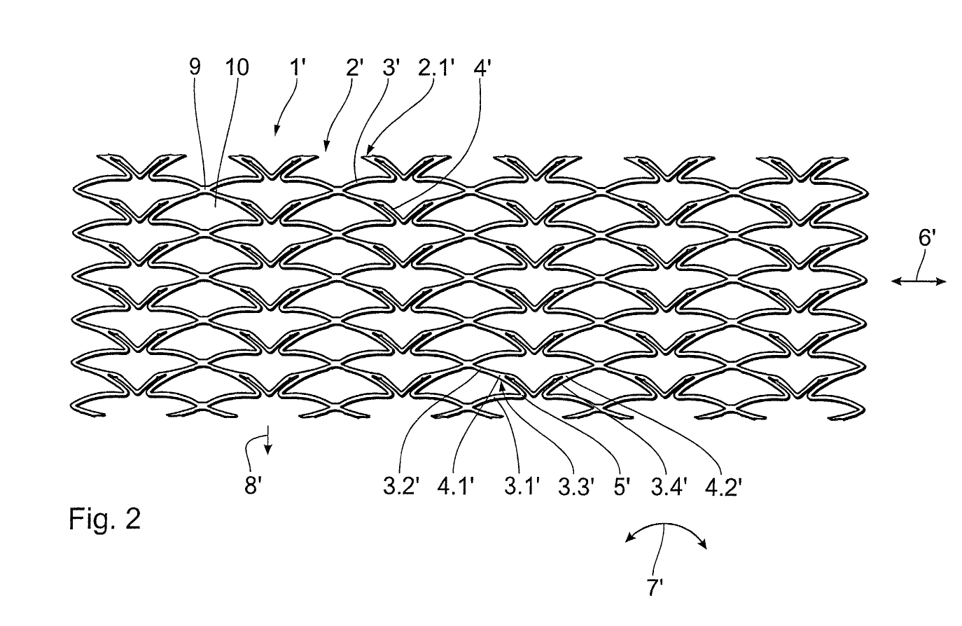

FIG. 2 is a plan view on to the development of the peripheral surface 1′ of a further embodiment of a stent according to the invention having a number of annular support portions 2′. The development of the peripheral surface 1′ is shown in the first condition of the stent in which it can be introduced into the blood vessel. In the illustrated example the stent consists exclusively of first support portions 2.1′ which are formed by bar elements 3′ extending in a meander configuration in the peripheral direction of the stent. The bar elements 3′ are connected together alternately by way of connecting bars 4′ and 9 in the longitudinal direction of the stent. It will be appreciated however that other variants may also involve a different sequence of the connecting bars 4′ and 9. In particular the stent may exclusively comprise connecting bars in the manner of the connecting bars 4′.

The bar element portions 3.1′ and 3.2′ adjoining a turning or reversal point 5′ are arranged in a V-shape in the illustrated first condition of the stent. They extend curvedly in a first direction indicated by the double-headed arrow 7′, in the longitudinal direction of the stent as indicated by the double-headed arrow 6′. By virtue of that curved, V-shaped arrangement of the bar element portions 3.1′, 3.2′ in the first condition of the stent, the bar elements 3′ themselves contribute to flexibility of the stent, in relation to its longitudinal direction.

If for example a compression force is applied to the bar elements 3′ in the longitudinal direction, the curvature of the bar element portions 3.1′ and 3.2′ increases, with the result that the bar element 3′ is reduced in length in the longitudinal direction of the stent. If conversely a pulling force is applied to the bar elements 3′ in the longitudinal direction, the curvature of the bar element portions 3.1′ and 3.2′ is reduced, with the result that the length of the bar element 3′ increases in the longitudinal direction of the stent. If therefore the stent is to be bent or curved, for example to follow a curved vessel configuration, then the bar elements 3′ are reduced in length in the longitudinal direction on the side of the stent which faces towards the center point of the curvature, by virtue of the compression force acting there, in the above-described manner, while, on its side remote from the associated center point of the curvature, the bar elements are increased in length in the longitudinal direction, by virtue of the pulling force acting there, in the above-described manner.

In the illustrated embodiment, the bar element portions 3.1′′and 3.2′ are of a continuous curvature which is uniform over the length thereof. That means that manufacture of the stent is particularly simple, by virtue of the simple geometry involved, with a desirable distribution of stresses over the bar element.

The curvature of the bar element portions 3.1′ and 3.2′ is so selected that the stresses which occur upon flexural deformation of the stent in relation to its longitudinal axis when it is being moved to the implantation location remain below the plastic deformation limit of the stent material. It will be appreciated that in other variants the width of the bar elements may also vary over the length thereof in order to achieve that effect.

In the illustrated embodiment the bar elements of all support portions 3′ are respectively curved in the same direction 7′. It will be appreciated however that in other variants a change in the direction of curvature of the bar element portions may also occur, between the support portions which are in mutually adjoining relationship in the longitudinal direction of the stent. It will further be appreciated that in a portion-wise manner the stent may also comprise bar elements of a different configuration, which do not involve the above-indicated properties.

The connecting bars 4′ are adapted and arranged to compensate for the reduction in length of the bar elements 3′ upon expansion of the stent into its second condition. For that purpose they are of a V-shaped configuration and respectively engage the central region 3.3 of the bar element portions 3.2′ and 3.4′ of two mutually adjoining bar elements 3′, wherein their engagement points 4.1′ and 4.2′ are at the same level in relation to a first peripheral direction 8′. Upon expansion of the stent therefore the spacing between those central regions 3.3′ of the bar elements 3′ does not change substantially, but the turning points 5′ only move closer to that central region 3.3′, in relation to the longitudinal direction. The reduction in length of the bar elements 3′ connected by way of the connecting bars 4′ in the longitudinal direction of the stent is thus compensated, upon expansion thereof.

The connecting bars 4′ are of a configuration adapted to the curvature of the bar element portions 3.2′ and 3.4′. In other words, in the first condition of the stent, they follow the configuration of the bar element portions 3.2′ and 3.4′ at a constant spacing over a long distance. That configuration also affords uniform distribution of the support locations for the vessel wall and thus good and uniform support for the vessel in the expanded condition of the stent.

The connecting bars 4′ further contribute to increasing the flexibility of the stent by virtue of their V-shaped configuration as their configuration means that relatively low levels of pulling or compression forces operative in the longitudinal direction of the stent, as a consequence of the long lever arm, already result in considerable deflections of the connecting bars 4 in the longitudinal direction of the stent.

The connecting bars 9 are in the form of short straight bars extending in the longitudinal direction of the stent between adjacent turning points of the bar elements 3′. They provide particularly stiff cells 10 which ensure good reliable radial support for the wall of the vessel.

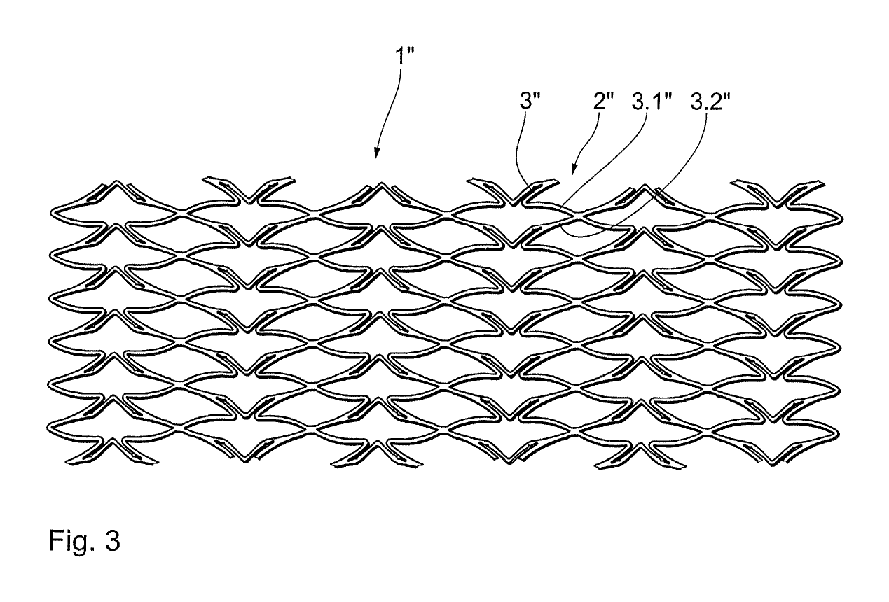

FIG. 3 shows a further embodiment of a stent according to the invention which corresponds in terms of its fundamental configuration and function to the embodiment of FIG. 2 so that here only the differences will be discussed.

One difference is that the peripheral surface 1″ comprises annular support portions 2″ comprising bar elements 3″′ in which the direction of curvature of the bar element portions 3.1″ and 3.2″ changes in the longitudinal direction of the stent. In this case, the two bar elements 3″ connected by way of a V-shaped connecting bar 4″ respectively have bar element portions 3.1″ and 3.2″ involving the same direction of curvature while the two bar elements 3″ connected by way of a short straight connecting bar 9″ respectively have bar element portions involving a different direction of curvature.

That configuration also results in the orientation of the connecting bars 4″ alternating in the longitudinal direction of the stent, with respect to the peripheral direction.

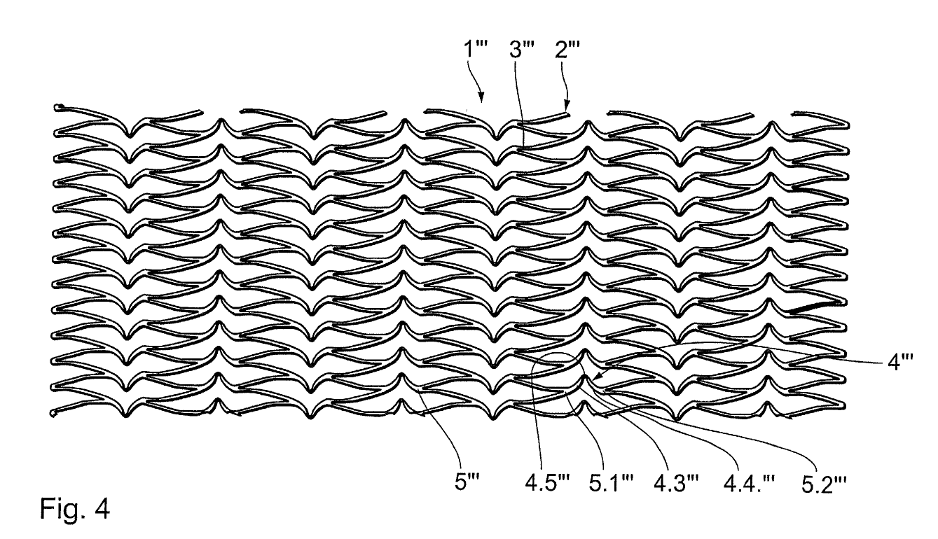

FIG. 4 shows a further embodiment of a stent according to the invention which corresponds in terms of the configuration and function of the bar elements 3″′ to the embodiments described with reference to the foregoing Figures, so here too only the differences will be discussed.

One difference is that the peripheral surface 1″′ comprises annular support portions 2″′ of bar elements 3″′ in which the direction of curvature of the bar element portions 3.1″ and 3.2″ changes in the longitudinal direction of the stent from one support portion to another.

A further difference is that there are provided V-shaped connecting bars 4″′ which engage the bar elements 3″′ in the region of mutually adjoining turning points 5.1″′ and 5.2″′ which are at the same level with respect to the peripheral direction. In this case, the limbs 4.3″′ and 4.4″′ of the connecting bar 4″′, starting from an arcuate root 4.5″′, go in an arcuate configuration into the bar element 3″′, with a continuous increase in width. In this case, upon expansion of the stent, as a consequence of the change in angle caused by deformation in the region of the respective turning point 5″′, there is a change in angle of the limbs 4.3″′ and 4.4″′, in such a way that the V-shaped connecting bar 4″′ is bent open, whereby its engagement points on the bar elements 3″′ move away from each other in the longitudinal direction of the stent and thus the reduction in length of the bar elements 3″′upon expansion of the stent is compensated.

In addition the described configuration of the connecting bars 4″′ contributes to increasing the flexibility of the stent in the manner set forth hereinbefore in relation to FIG. 2.

In the illustrated embodiment each turning point 5″′ is engaged by a connecting bar 4″′, while the connecting bars 4″′ are of an orientation which alternates in the longitudinal direction of the stent, with respect to the peripheral direction. It will be appreciated however that the other variants may also have fewer connecting bars, so that for example there may be regular gaps without connecting bars between the turning points of the bar elements. Furthermore it will also be appreciated that there may be a different sequence in regard to the orientation of the connecting bars, with respect to the peripheral direction.

What is claimed is:

1. A stent, in particular a coronary stent, for expansion from a first condition into an expanded second condition in which it holds a vessel in an expanded state in an implantation location, comprising:
a tubular body with a peripheral surface formed from a number of annular support portions that comprise bar elements connected in a longitudinal direction of the stent at an engagement point by way of connecting bars;
wherein the bar elements of at least a first annular support portion extend in a meander configuration in a peripheral direction of the stent and a number of bar element portions which adjoin a turning point are arranged in a V-shape in the first condition of the stent, characterised in that the bar element portions of the first annular support portion extend curvedly in a first direction in the longitudinal direction of the stent.

2. The stent of claim 1, wherein:
the bar element portions are of a continuously curved configuration.

3. The stent of claim 2, wherein:
the bar element portions are curved uniformly over a length thereof.

4. The stent of claim 1, wherein:
the bar element portions are curved uniformly over a length thereof.

5. The stent of claim 4, wherein:
the bar element portions are curved in such a way and/or a width of the bar elements varies over a length thereof in such a way that the stresses which occur upon flexural deformation of the stent with respect to a longitudinal axis thereof upon being moved to the implantation location remain below a plastic deformation limit of a material comprising the stent.

6. The stent of claim 3, wherein:
the bar element portions are curved in such a way and/or a width of the bar elements varies over a length thereof in such a way that the stresses which occur upon flexural deformation of the stent with respect to a longitudinal axis thereof upon being moved to the implantation location remain below a plastic deformation limit of a material comprising the stent.

7. The stent of claim 1, wherein:
the bar element portions are curved in such a way and/or a width of the bar elements varies over a length thereof in such a way that the stresses which occur upon flexural deformation of the stent with respect to a longitudinal axis thereof upon being moved to the implantation location remain below a plastic deformation limit of a material comprising the stent.

8. The stent of claim 7, further comprising:
a number of adjacent first annular support portions whose bar element portions are curved in the same direction.

9. The stent of claim 6, further comprising:
a number of adjacent first annular support portions whose bar element portions are curved in the same direction.

10. The stent of claim 5, further comprising:
a number of adjacent first annular support portions whose bar element portions are curved in the same direction.

11. The stent of claim 7, further comprising:
a number of adjacent first annular support portions, wherein the direction of curvature of the bar element portions of the annular support portions changes in the longitudinal direction of the stent.

12. The stent of claim 6, further comprising:
a number of adjacent first annular support portions, wherein the direction of curvature of the bar element portions of the annular support portions changes in the longitudinal direction of the stent.

13. The stent of claim 5, further comprising:
a number of adjacent first annular support portions, wherein the direction of curvature of the bar element portions of the annular support portions changes in the longitudinal direction of the stent.

14. The stent of claim 11, wherein:
the direction of curvature of the bar element portions changes from one annular support portion to another or the annular support portions have bar element portions in pairs with the same direction of curvature.

15. The stent of claim 12, wherein:
the direction of curvature of the bar element portions changes from one annular support portion to another or the annular support portions have bar element portions in pairs with the same direction of curvature.

16. The stent of claim 13, wherein:
the direction of curvature of the bar element portions changes from one annular support portion to another or the annular support portions have bar element portions in pairs with the same direction of curvature.

17. The stent of claim 16, wherein:
the connecting bars compensate for the reduction in length of the bar elements in the longitudinal direction of the stent upon expansion of the stent.

18. The stent of claim 15, wherein:
the connecting bars compensate for the reduction in length of the bar elements in the longitudinal direction of the stent upon expansion of the stent.

19. The stent of claim 14, wherein:
the connecting bars compensate for the reduction in length of the bar elements in the longitudinal direction of the stent upon expansion of the stent.

20. The stent of claim 10, wherein:
the connecting bars compensate for the reduction in length of the bar elements in the longitudinal direction of the stent upon expansion of the stent.

21. The stent of claim 9, wherein:
the connecting bars compensate for the reduction in length of the bar elements in the longitudinal direction of the stent upon expansion of the stent.

22. The stent of claim 8, wherein:
the connecting bars compensate for the reduction in length of the bar elements in the longitudinal direction of the stent upon expansion of the stent.

23. The stent of claim 1, wherein:
the connecting bars compensate for the reduction in length of the bar elements in the longitudinal direction of the stent upon expansion of the stent.

24. The stent of claim 23, wherein:
the engagement points and the length of the connecting bars are so selected that the reduction in length of the bar elements in the longitudinal direction of the stent upon expansion of the stent is substantially compensated.

25. The stent of claim 22, wherein:
the engagement points and the length of the connecting bars are so selected that the reduction in length of the bar elements in the longitudinal direction of the stent upon expansion of the stent is substantially compensated.

26. The stent of claim 21, wherein:
the engagement points and the length of the connecting bars are so selected that the reduction in length of the bar elements in the longitudinal direction of the stent upon expansion of the stent is substantially compensated.

27. The stent of claim 20, wherein:
the engagement points and the length of the connecting bars are so selected that the reduction in length of the bar elements in the longitudinal direction of the stent upon expansion of the stent is substantially compensated.

28. The stent of claim 19, wherein:
the engagement points and the length of the connecting bars are so selected that the reduction in length of the bar elements in the longitudinal direction of the stent upon expansion of the stent is substantially compensated.

29. The stent of claim 18, wherein:
the engagement points and the length of the connecting bars are so selected that the reduction in length of the bar elements in the longitudinal direction of the stent upon expansion of the stent is substantially compensated.

30. The stent of claim 17, wherein:
the engagement points and the length of the connecting bars are so selected that the reduction in length of the bar elements in the longitudinal direction of the stent upon expansion of the stent is substantially compensated.

31. The stent of claim 30, wherein:
the connecting bars are rectilinear and extend between two mutually facing turning points of two adjoining bar elements that are displaced relative to each other in the first condition of the stent in the peripheral direction of the stent by between once and twice the period.

32. The stent of claim 29, wherein:
the connecting bars are rectilinear and extend between two mutually facing turning points of two adjoining bar elements that are displaced relative to each other in the first condition of the stent in the peripheral direction of the stent by between once and twice the period.

33. The stent of claim 28, wherein:
the connecting bars are rectilinear and extend between two mutually facing turning points of two adjoining bar elements that are displaced relative to each other in the first condition of the stent in the peripheral direction of the stent by between once and twice the period.

34. The stent of claim 27, wherein:
the connecting bars are rectilinear and extend between two mutually facing turning points of two adjoining bar elements that are displaced relative to each other in the first condition of the stent in the peripheral direction of the stent by between once and twice the period.

35. The stent of claim 26, wherein:
the connecting bars are rectilinear and extend between two mutually facing turning points of two adjoining bar elements that are displaced relative to each other in the first condition of the stent in the peripheral direction of the stent by between once and twice the period.

36. The stent of claim 25, wherein:
the connecting bars are rectilinear and extend between two mutually facing turning points of two adjoining bar elements that are displaced relative to each other in the first condition of the stent in the peripheral direction of the stent by between once and twice the period.

37. The stent of claim 24, wherein:
the connecting bars are rectilinear and extend between two mutually facing turning points of two adjoining bar elements that are displaced relative to each other in the first condition of the stent in the peripheral direction of the stent by between once and twice the period.

38. The stent of claim 1, wherein:
the connecting bars are rectilinear and extend between two mutually facing turning points of two adjoining bar elements that are displaced relative to each other in the first condition of the stent in the peripheral direction of the stent by between once and twice the period.

39. The stent of claim 38, wherein:
the connecting bars are adapted to increase the flexibility of the stent.

40. The stent of claim 37, wherein:
the connecting bars are adapted to increase the flexibility of the stent.

41. The stent of claim 36, wherein:
the connecting bars are adapted to increase the flexibility of the stent.

42. The stent of claim 35, wherein:
the connecting bars are adapted to increase the flexibility of the stent.

43. The stent of claim 34, wherein:
the connecting bars are adapted to increase the flexibility of the stent.

44. The stent of claim 33, wherein:
the connecting bars are adapted to increase the flexibility of the stent.

45. The stent of claim 32, wherein:
the connecting bars are adapted to increase the flexibility of the stent.

46. The stent of claim 31, wherein:
the connecting bars are adapted to increase the flexibility of the stent.

47. The stent of claim 1, wherein:
the connecting bars are adapted to increase the flexibility of the stent.

48. The stent of claim 47, wherein:
the connecting bars are V-shaped.

49. The stent of claim 46, wherein:
the connecting bars are V-shaped.

50. The stent of claim 45, wherein:
the connecting bars are V-shaped.

51. The stent of claim 44, wherein:
the connecting bars are V-shaped.

52. The stent of claim 43, wherein:
the connecting bars are V-shaped.

53. The stent of claim 42, wherein:
the connecting bars are V-shaped.

54. The stent of claim 41, wherein:
the connecting bars are V-shaped.

55. The stent of claim 40, wherein:
the connecting bars are V-shaped.

56. The stent of claim 39, wherein:
the connecting bars are V-shaped.

57. The stent of claim 56, wherein:
the connecting bars engage a central region of the bar element portions and are adapted to the curvature thereof.

58. The stent of claim 55, wherein:
the connecting bars engage a central region of the bar element portions and are adapted to the curvature thereof.

59. The stent of claim 54, wherein:
the connecting bars engage a central region of the bar element portions and are adapted to the curvature thereof.

60. The stent of claim 53, wherein:
the connecting bars engage a central region of the bar element portions and are adapted to the curvature thereof.

61. The stent of claim 52, wherein:
the connecting bars engage a central region of the bar element portions and are adapted to the curvature thereof.

62. The stent of claim 51, wherein:
the connecting bars engage a central region of the bar element portions and are adapted to the curvature thereof.

63. The stent of claim 50, wherein:
the connecting bars engage a central region of the bar element portions and are adapted to the curvature thereof.

64. The stent of claim 49, wherein:
the connecting bars engage a central region of the bar element portions and are adapted to the curvature thereof.

65. The stent of claim 48, wherein:
the connecting bars engage a central region of the bar element portions and are adapted to the curvature thereof.

66. A catheter for stent implantation having a stent, for expansion from a first condition into an expanded second condition in which it holds a vessel in an expanded state in an implantation location, wherein the stent comprises:
a tubular body with a peripheral surface formed from a number of annular support portions that comprise bar elements connected in a longitudinal direction of the stent at an engagement point by way of connecting bars;
wherein the bar elements of at least a first annular support portion extend in a meander configuration in a peripheral direction of the stent and a number of bar element portions which adjoin a turning point are arranged in a V-shape in the first condition of the stent, characterised in that the bar element portions of the first annular support portion extend curvedly in a first direction in the longitudinal direction of the stent.
